(19) United States
(12) Patent Application Publication
(10) Pub. No.: US 2021/0361531 A1
(43) Pub. Date: Nov. 25, 2021

(54) DIGITALISED, PORTABLE AND CONSOLIDATED APPARATUS AND METHOD FOR DISPENSING, TRACKING AND COMMUNICATING MULTIPLE-DOSE ADHERENCE IN REAL-TIME AND INCLUSIVE OF REFILL MECHANISM

(71) Applicant: Gauri SHANKAR ANGRISH, New Delhi (IN)

(72) Inventor: Gauri SHANKAR ANGRISH, New Delhi (IN)

(21) Appl. No.: 16/634,159

(22) PCT Filed: Jul. 26, 2018
(86) PCT No.: PCT/IN2018/050492

§ 371 Date: Jan. 26, 2020

(30) Foreign Application Priority Data
Jul. 26, 2017 (IN) 201711026542

(51) Int. Cl. A61J 7/04; G16H 40/63; G16H 20/13

(52) U.S. Cl. CPC A61J 7/049; A61J 7/0427; A61J 2200/70; G16H 20/13; A61J 2200/30; G16H 40/63

(57) ABSTRACT

This invention relates to a safer and more comprehensive, economical, convenient and digitalised apparatus and method that equips the existing multiple-dose dispenser with a unique assembly configured to enable additional capabilities including the automated dispensing of multiple-dose pouches containing individual units of homogenous and/or heterogeneous pharmaceutical products which are retained in and/or without manufacturer's packaging, in exact customised quantities and combinations, exactly according to the dose time prescribed by the doctor, with optional automated light and/or sound alerts when the relevant dose pouches are dispensed, provision of a button which when pressed by the user cuts the dispensed pouches and stores and/or communicates the time of cutting with the central systems in real-time. The invention also contains features such as compatibility with phone applications to allow the user to control the device for allowing dispensed pouches to go back in and reporting emergencies and/or adverse drug reactions.

The following specification particularly describes the invention and the manner in which it is to be performed.

### FIELD OF INVENTION

This invention relates to a safer and more comprehensive, economical, convenient and digitalised apparatus and method that equips the existing multiple-dose dispenser (Application No. 4321/DEL/2015, the “Existing Dispenser”) with a unique assembly configured to enable additional capabilities including the automated dispensing of multiple-dose pouches containing individual units of homogenous and/or heterogeneous pharmaceutical products which are retained in and/or without manufacturer's packaging, in exact customised quantities and combinations, exactly according to the dose time prescribed by the doctor, with optional automated light and/or sound alerts when the relevant dose pouches are dispensed, provision of a button which when pressed by the user cuts the dispensed pouches and stores and/or communicates the time of cutting with the central systems in real-time. The invention also contains features such as compatibility with IOS and Android phone applications to allow the user to control the device via his/her phone, computer or tablet, additional buttons for allowing dispensed pouches to go back in and reporting emergencies and/or adverse drug reactions. The invention ensures that the user takes his/her doses exactly according to the prescription and tracks and communicates the users adherence or consumption patterns in real-time to improve his/her treatment outcomes.

### BACKGROUND OF THE INVENTION

There are various devices known in the art which dispense medication and track dose adherence, including in real-time. The prior art discloses a number of devices that assist with the dispensation of prescribed medication and record the medical treatment progress. However, the functionality and effectiveness of the medicine/pharmacy management systems in the existing art is limited due to their inability to (i) clearly notify the patient to consume the prescribed medication at the specified time; (ii) alert the patient to charge the device when required; (ii) ensure that medicines are dispensed in the exact quantities and/or combinations as prescribed by the doctor; (iv) allow remote access and control over the features and functions of the device; (v) enable a sufficiently user-friendly and uncomplicated interface; and (vi) account for medicines other than solid pills, thereby excluding from the medicine/pharmacy management system all injections, syrups, powders and other non-oral solids and liquids and/or oral liquids that may be part of a consumer's prescribed treatment. Further, the prior art does not enable the dispensing of pharmaceutical substances other than open pills (i.e. from which the manufacturer's packaging has been removed). In such a situation there is always a risk of that the dispensed medicines have been contaminated in some manner due to exposure to other medicines, moisture or humidity, and have consequently lost some of their efficacy and pharmacological properties. Accordingly, there is a need to obviate the lacunae in the prior art and provide safer, simpler and more user-friendly and efficient tracking and monitoring solutions that enable a convenient, cost-effective and portable system of multiple-dose adherence that is both safe and easy for consumer use.

U.S. Pat. No. 5,582,323A relates to a medication dispenser and monitor which includes a housing containing multiple pill dispensing compartments for dispensing medication to a patient at a desired time. The amount of medication required by the patient per dose is loaded into each individual compartment. The device is programmed to dispense each pill dispensing compartment at a predetermined time corresponding to the time in which the patient is supposed to take the medication. The dispensed medication is received by a tray providing access to the patient. If the patient does not open the tray, audio and visual alarms are activated. In addition, the processor initiates a communication with a central monitoring station through the phone lines to notify the station of the non-compliance. The invention also contacts the emergency personnel through phone lines and initiates two-way hands free communication between the patient and emergency personnel. The invention further includes a pendant transmitter worn by the patient to contact emergency personnel. A limitation of this invention is that it tracks the opening of a tray, which may not necessarily be done only at the time of consumption by the patient and, accordingly, may not be indicative of actual consumption.

US20140052468A1 relates to a medication adherence and monitoring system which also enables dose reminder notifications. The medication adherence system herein includes data-enabled pharmaceutical containers, which provide mechanisms for reminding at dose time, then tracking and communicating valid dose events. The containers have a built-in dose detection algorithm, wherein a dose event is tracked as valid based on sensing that the data-enabled pharmaceutical container is both opened and tilted past a predefined minimum threshold angle simultaneously for a predefined minimum amount of time.

U.S. Pat. No. 7,369,919B2 comprises of a medication tray with containers filled with medicines and a docking station. The medication tray receives prescription data when it receives the medicine containers, data in respect of which is simultaneously downloaded to the docking station. The docking station monitors and reports to third parties, via a network, a patient's compliance with various medication treatment regimens. Medication containers are provided with low-bit tags that provide container presence information to the docking station. The docking station provides visual and/or audio signals regarding prescription data to a patient.

U.S. Pat. No. 5,200,891A relates to a device for monitoring a patient's medication and prompting the patient into certain medication taking schedule and/or certain programming steps and routines. The device has various compartments, each of which stores medication and an electrical signaling system to emit medication alert signals from time to time. There are separate signals to indicate that medication should be taken, the compartment from which the medication should be taken and in what quantity. If a designated compartment is not opened and closed within a predetermined period of time, the electrical signaling system will sound an alarm. If each designated compartment is opened and closed, the take-medication signal and the alarm (if operating) are turned off and the event is recorded for later review.

U.S. Pat. No. 6,085,752A relates to a portable electronic medical device that comprises of separate keys, buttons or touch points on a screen associated with each medication or disease. Each of the keys facilitates access to information related to the associated medication or disease. When the patient presses a key, the device displays a prescription label message concerning the medication contained in the medication compartment associated with the actuated key. Further successive actuations of a same dedicated key will access additional information concerning the medication contained in the medication compartment associated with the actuated key, and this may include information about the particular medical condition that the medication treats. There is a compartment tray of variable capacity and storage system which provides for pre-loading, and pre-labeling of medications, so that when the patient exhausts a supply of medication in one tray, a second pre-loaded and pre-labeled tray can replace the exhausted tray. Opening and closing a medication compartment in the tray signals the tracking function of the device. There are is a specific to indicate that certain scheduled medication doses are due to be taken, and as separate general alert that other actions are required (such as responding to certain patient queries, performance of a particular test, placing the device within the cradle for communication with a remote device, phoning or making an appointment with the health care provider, giving an injection, inhaling an aerosolized medication). Further, there is a graphic display to indicate the patient's progress. The device either automatically presents the supply-to-load messages, or presents the supply-to load messages when the patient presses a dedicated key for a particular medication.

US20130218588A1 includes smart tray monitors and reports to third parties a patient's compliance with various medication treatment regimens. Medication containers are provided with electromagnetic tags that provide various information about medicament contained within a respective container. The smart tray is equipped with a processor and reader that interrogates each respective electromagnetic tag to identify medicament(s) contained within each container. Using the retrieved information, the smart tray provides visual and/or audio signals to a patient to remind the patient when and how much of various medicaments to take. The smart tray also monitors, via the reader, when a medication container is removed. The smart tray can communicate with one or more third parties, such as healthcare providers, pharmacies, and other suppliers of healthcare products and services via a computer network. The smart tray can also communicate with various appliances and can modify medication regimens for particular medicaments in response to data received from various appliances.

A number of limitations present in the prior art are as follows:
1. Devices and methods for medicine tracking and adherence in the prior art dispense only open pills i.e. oral solids (i.e. tablets and capsules) that are not contained in manufacturers' packaging. It is relevant that the removal of manufacturer's packaging and the storage of such open medicines over several days can result in the loss of their efficacy and pharmacological properties. Further, medicines are susceptible to alteration as a result of their exposure to heat, light and interaction with other medicines, and such reactions adversely affect the safety of the medicines. Further, the retail sale of open pharmaceutical products and such re-packaged medication is not permitted under applicable law in India and various other countries. The present invention ensures that the safety, efficacy and pharmacological properties of each medicine is retained by dispenses medicines retaining the manufacturer's original packaging at the time of consumption.
2. Medicine tracking and adherence in the prior art entails complicated devices and processes involving a multitude of units. The patient is required to either manually retrieve medication from different compartments and quantities, or the desired dosage is assembled pursuant to a multiplicity of instructions for each unit from a database immediately preceding consumption, which could potentially result in mix-ups of medication. Further, the patient alerts do not notify or ensure the consumption of medicines in the exact quantities and combinations, as prescribed. The present invention, however, dispenses a pre-organised dosage pack which comprises of the medication a patient is required to consume in exact combinations and quantities as prescribed, and also comes with dosage instructions for the patient. Further, it allows for easy adoption by consumer/patients by dispensing dose pouches with labels in various languages. This enables a safer and more patient-friendly and efficient medicine management system for the consumer.
3. The tracking functions in the prior art are carried out by way of complicated procedures and actions (such as tilting at particular angles, opening and closing of trays and compartments, etc.) which may be amenable to errors. However, the present invention ensures a more reliable tracking mechanism through the simple action of cutting, which is also more user-friendly and geriatric-friendly.
4. Since devices and methods for medicine tracking and adherence in the prior art do not take into account non-oral pills and drugs and/or oral liquids/powder/injections, the pharmacy/medicine management system rendered thereby is neither comprehensive nor complete. The present invention, however, provides a comprehensive system for management of all types of substances such as drugs/medicines by dispensing pouches with FLAT TABS, cardboard or any other material representatives of those substances that cannot be pouched or enveloped.
5. The prior art does not allow remote access to, or control of, the features of the devices. However, the features in the present invention can be accessed and controlled remotely via the linked iOS or Android application therein, and this enables a more user-friendly and efficient dose adherence system.
6. The existing devices are also very exorbitantly priced, in comparison to which the medicine management system relating to the present invention is considerably more economically viable solution.

### OBJECT OF PRESENT INVENTION

An object of the present invention is to ensure that the user takes his/her doses exactly according the prescribed time by automatically dispensing the multi-dose pouches of the relevant dose at the prescribed time and automatically alerting the user with light and/or sound on the body of the apparatus.

Another objective of the present invention is to allow the user to conveniently cut the automatically dispensed dose pouches by pressing a button present on the apparatus so that they can consume the contents of the said pouch.

Another objective of the present invention is to track the time at which the user cuts the dispensed dose pouches using the button present on the apparatus. This information results in the user's adherence or consumption patterns and, when compared with the prescribed dosage times of the user, gives an adherence trend/score card which can be utilised by the user's doctor or healthcare professional to map with the user's treatment progression.

Another objective of the present invention is to communicate the user's non-adherence or non-consumption of each dose in real-time to allow relevant actions to be taken. If a user does not cut the relevant dose pouches at the prescribed time then an alert is sent to his chosen caregivers, family members and/or doctor so specific actions can be taken to ensure that the patient cuts and consumes the dose closest to the prescribed time, thus maintaining complete adherence.

Another objective of the present invention is to allow the users to alert chosen caregivers, family members and/or doctors about any emergencies or adverse drug reactions. A button is provided on the apparatus, which when pressed by the user, sends an alert to the user's chosen persons about the event so that appropriate action can be taken thereafter.

Another objective of the present invention is to allow the user to record when they have consumed the medicines from the dispensed and cut the dose pouch. A button is provided on the apparatus, which when pressed by the user, records the time stamp for the time on which they have consumed the said dose.

Another objective of the present invention is to allow remote access and control of all the features via the linked iOS or Android application. The application allows an individual to dispense pouches for a specific dose or for multiple doses or days, retrieve pouches back into the apparatus incase they have dispensed more than needed and cut the dispensed pouches.

Another objective of the present invention is to automatically share data with the central software regarding current battery life and number of pouches left in the dispenser allowing the system to manage change or recharge of battery and management of dose refills respectively.

Another objective of the present invention is to enable the dispensation of all types of homogenous and/or heterogeneous pharmaceutical products and oral solids such as pills, which are either retained in manufacturer's packaging or otherwise, in the exact quantities and combinations as prescribed. This is a significant aspect of medicine management since dispensing medicines retained in manufacturers' packaging ensures safety, efficacy and pharmacological properties of enveloped substances.

Another objective of the present invention is to allow the user to charge their device via a simple USB port charger.

### BRIEF DESCRIPTION OF ACCOMPANYING DRAWINGS

The following drawings are illustrative of particular embodiments for enabling the system and method of the present invention and are not intended to limit the scope of the invention. The drawings are not to scale (unless so stated) and are intended for use in conjunction with the explanations in the following detailed description.

FIG. 1 is an exemplary representation of apparatus for the automatic dispensing unit comprising of a motor (1) and rollers (2) connected by gears (5) along with an IR sensor (3). The figure also shows a representation of the dose pouch (4) to highlight the action of the dispensing unit.

FIG. 2 is an exemplary representation of the apparatus for the cutting unit comprising of a blade (1) and blade holder (2) mounted on a lead screw (3), powered by a motor (4) which is connected by gears. The blade holder also includes an leveler (5) that holds the pouch down while the blade cuts.

FIG. 3 exemplary representation of the apparatus of the side view of the arrangement of the section of the apparatus that holds the PCB (1) and the battery (2).

FIG. 4 exemplary representation of the apparatus for the arrangement of the buttons with multiple functionalities including cutting (1), dispensing/retrieving (2), consumption tracking (3) and emergency alert (4) along with the USB port (5) for charging and alert light (6) for alerting user about dose time and low battery.

### DETAILED DESCRIPTION OF THE EMBODIMENTS

### Description

The present invention relates to a digitalised apparatus and method with refill mechanism for dispensing, tracking and communicating multiple-dose adherence in real-time. The said apparatus comprises of:

a dispensing unit, comprising of motors and rollers, for automatically dispensing and retrieving relevant dose pouches, when needed;

a cutting unit, comprising of motors, a blade with blade holder and a lead screw, for cutting the dispensed dose pouches, once the user presses the relevant button;

an IR sensing unit that senses the eye marks present on the dispensed and cut pouches, thus maintaining a count;

a PCB (printed circuit board) that includes circuitry for multiple communication technologies including GSM, WiFi and Bluetooth;

a battery that provides power for the said actions to be carried out;

a USB charging port that allows for recharging of battery;

a first button with light that allows user to activate the cutting unit;

a second button that allows user to send alert via inbuilt communication technologies in case of emergencies;

a third button that allows user to dispense additional pouches and/or retrieve the dispensed pouches;

a fourth button that allows user to record when they have consumed the medicines from the dispensed and cut dose pouch; and

alert lights for dose reminders and/or low battery.

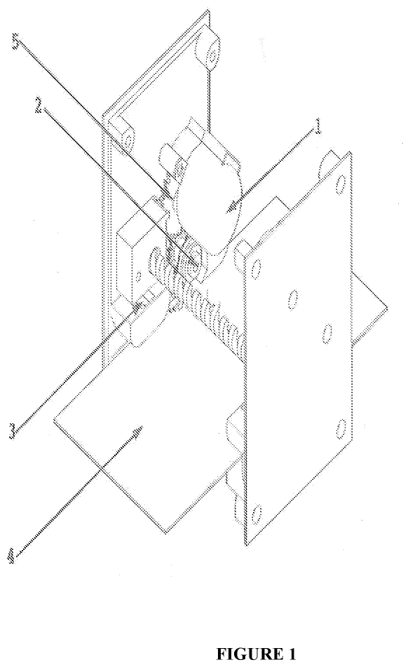

A schematic representation of a system where the present invention may be implemented is illustrated in FIG. 1. FIG. 1 illustrates the dose pouch (4) that once fed into the apparatus is automatically moved ahead and dispensed by the rollers (2) powered by the motor (1). The motor is able to power the rollers using connected gears (5). As the dose pouches (4) are moved ahead, they pass through the IR sensors (3), which reads the eye marks on the dose pouches (4) and uses that to calculate count of those getting dispensed.

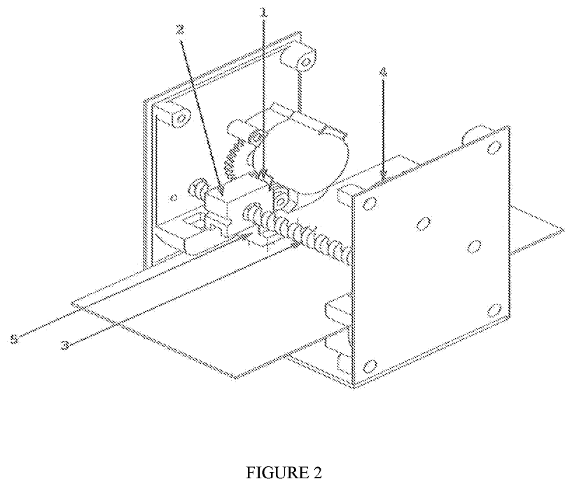

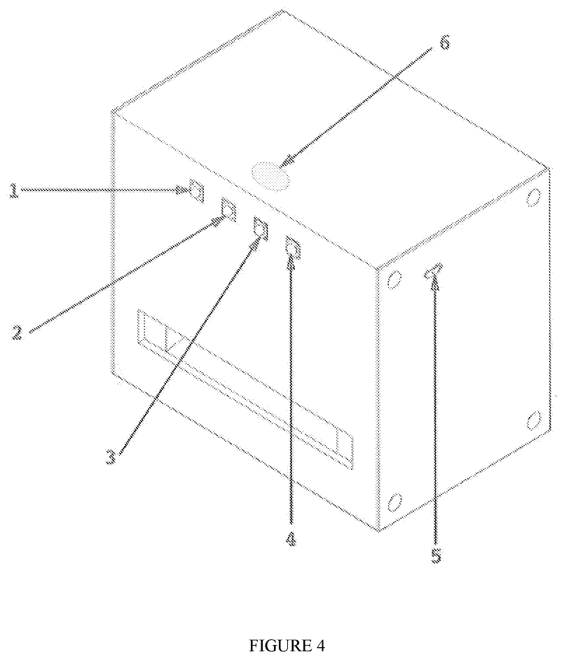

FIG. 2 illustrates the cutting mechanism. Once the dose pouch has been dispensed by the mechanism explained in FIG. 1, the user has to press a button as explained in FIG. 4 to activate the cutting mechanism. Once the button is pressed, the lead screw (3) powered by the motor (4) starts to rotate and thus moves the blade (1), blade holder (2) and leveler (5) across the width of the dose pouch. The leveler (5) hold the section of the pouch down while the blade (1) cuts through it, thus detaching the pouch from the rest.

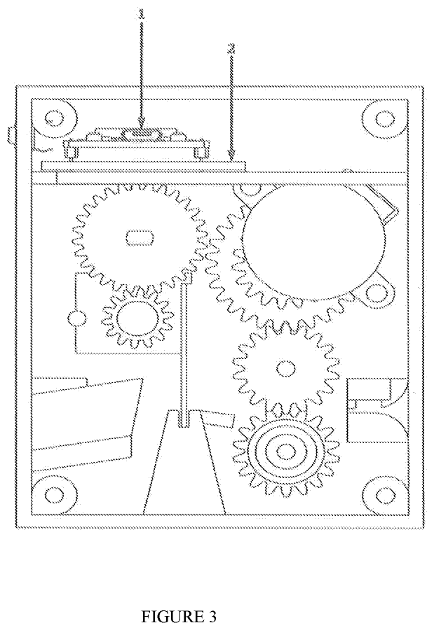

FIG. 3 illustrates the part of the apparatus that houses the battery (2) that provides power to all mechanisms and PCB (1) that contains the circuitry, processor and communication modules including those for GSM, Wifi and Bluetooth that allow the device to communicate with other systems. The processor downloads and gives the command to the dispensing mechanism explained in FIG. 1 to be able to dispense the relevant dose pouches at the correct time, tracks the time at which the user cuts the dose pouches using the cutting mechanism as explained in FIG. 2 and communicates this time stamp to other systems using the aforementioned communication options

FIG. 4 illustrates the arrangement of the buttons that when pressed carry out the relevant command. Once the relevant dose pouches have been automatically dispensed by the dispensing mechanism as mentioned in FIG. 1, the cutting button (1) lights up, alerting the user to press the same. Once this cutting button (1) is pressed by the user, the cutting mechanism as described in FIG. 2 is activated and the timestamp of that action is stored and communicated as described in FIG. 3. The dispensing/retrieving button (2) when pressed activates the dispensing mechanism as explained in FIG. 1 and allows the user to dispense and/or retrieve dose pouches on demand. The emergency alert button (4) when pressed by the user sends an alert to connected systems which in turn alert relevant persons to take appropriate actions. The USB port (5) present on the apparatus allows the user to charge the device and the alert light (6) informs the user when it is time to cut the dose pouches that have been automatically dispensed or when the apparatus has low battery and needs to be charged.

I claim:

1. Apparatus and method for dispensing, tracking and communicating multiple-dose adherence in real-time by way of a digitalised, portable and consolidated device inclusive of refill mechanism, which comprises of:
a dispensing unit for automatically dispensing and retrieving relevant dose pouches as required;
a cutting unit for cutting the dispensed dose pouches, once the user presses the relevant button;
an IR sensing unit that senses the eye marks present on the dispensed and cut pouches, thereby maintaining a count;
a printed circuit board for multiple communication technologies including GSM, WiFi and Bluetooth;
a battery that provides power for the aforesaid actions to be carried out;
a USB charging port that allows for recharging the battery;
a first button with light that allows the user to activate the cutting unit;
a second button that allows alerts to be sent by way of in-built communication mechanisms in case of emergencies;
a third button that allows the dispensation of additional pouches and/or retrieval of the dispensed pouches;
a fourth button that allows the recording of consumption of medicines dispensed in exact prescribed quantities and combinations; and
alert lights for dose reminders and/or low battery.

2. Apparatus and method for dispensing, tracking and communicating multiple-dose adherence in real-time by way of a digitalised, portable and consolidated device inclusive of refill mechanism in claim 1, which includes

3. Apparatus and method for dispensing, tracking and communicating multiple-dose adherence in real-time by way of a digitalised, portable and consolidated device inclusive of refill mechanism in claim 1, wherein the dispensing unit includes a motor and rollers, connected by gears, and/or an IR sensor

4. Apparatus and method for dispensing, tracking and communicating multiple-dose adherence in real-time by way of a digitalised, portable and consolidated device inclusive of refill mechanism in claim 1, wherein the cutting unit includes one or more motors, one or more blades with one or more blade holders and one or more lead screws, and may also contain one or more motors and gears.

5. Apparatus and method for dispensing, tracking and communicating multiple-dose adherence in real-time by way of a digitalised, portable and consolidated device inclusive of refill mechanism in claim 1, which includes an IR sensing unit.

6. Apparatus and method for dispensing, tracking and communicating multiple-dose adherence in real-time by way of a digitalised, portable and consolidated device inclusive of refill mechanism in claim 1, wherein the PCB (printed circuit board) includes circuitry for multiple communication technologies including GSM, WiFi and Bluetooth.

7. Apparatus and method for dispensing, tracking and communicating multiple-dose adherence in real-time by way of a digitalised, portable and consolidated device inclusive of refill mechanism in claim 1, which includes a battery and/or USB charging port.

8. Apparatus and method for dispensing, tracking and communicating multiple-dose adherence in real-time by way of a digitalised, portable and consolidated device inclusive of refill mechanism in claim 1, which includes multiple buttons which allow users to
a. activate the cutting unit;
b. send alerts via inbuilt communication technologies when required;
c. dispense additional pouches and/or retrieve the dispensed pouches;
d. dispense additional pouches and/or retrieve the dispensed pouches; and
e. alert users for dose reminders and/or low battery.
